(19) United States
(12) Patent Application Publication
(10) Pub. No.: US 2008/0071648 A1
(43) Pub. Date: Mar. 20, 2008

(54) Integrated control system and method for automatic medicine packaging apparatuses

(76) Inventor: Jun Ho Kim, Taegu (KR)

Correspondence Address: PARK LAW FIRM, 3255 WILSHIRE BLVD, SUITE 1110, LOS ANGELES, CA, 90010, US

(21) Appl. No.: 11/601,262

(22) Filed: Nov. 17, 2006

(30) Foreign Application Priority Data
Sep. 20, 2006 (KR) 10-2006-0091294

(51) Int. Cl. G06Q 10/00

(52) U.S. Cl. 705/28

(57) ABSTRACT

An integrated control system for controlling a plurality of automatic tablet packaging apparatuses, includes an operation unit that receives operation signal input for performing functions, a communication unit that is adapted to send/ receive data to and from the automatic tablet packaging machines and a microprocessor that stores programs that perform communication with the automatic tablet packaging apparatuses and managing tablet inventory and expendable items, controls data communication by the communication unit, and controls display of operation status of a particular automatic table packaging apparatus. The operation unit selects one of the automatic table packaging apparatuses for which checking status is required, and receives operation signal input for checking detailed status for the appointed apparatus. The integrated control system communicates with a warehouse management system and performs managing tablets and expendable items stored in a warehouse.

### CLAIMING FOREIGN PRIORITY

The applicant claims and requests a foreign priority, through the Paris Convention for the Protection of Industrial Property, based on patent applications filed in the Republic of Korea (South Korea) with the filing date of Sep. 20, 2006 with the patent application number 10-2006-0091294 by the applicant, the contents of which are incorporated by reference into this disclosure as if fully set forth herein.

### BACKGROUND OF THE INVENTION

The present invention relates to an integrated control system and method for automatic medicine packaging apparatuses. More particularly, this invention relates to an integrated control system and method for automatic medicine packaging apparatuses by which a plurality of packaging apparatuses can be integrally managed and controlled with a single system.

Generally, an automatic medicine packaging apparatus stores medicines classified in a plurality of cassettes, and discharges tablets according to a prescription of a doctor and packs the tablets per dose. The cassettes are installed above tablet feeders. Each cassette is separately controlled to discharge a tablet that it contains with a microprocessor. The discharged tablet falls through passages provided in the tablet feeder. The tablets are collected into a hopper and then the collected tablets are discharged downward from the hopper and sealed with a sealing device and sealing envelopes, on which administering instructions are printed.

Pharmaceutical practice in Europe shows an example for need of integrally controlling multiple automatic medicine packaging machines. In Europe, a medicine supply service is designated for each local area. Such service is equipped with tens of automatic medicine packaging machines connected one another, each of which packs medicines according to prescriptions provided by hospitals. Two or three pharmacists manage the entire machines and individual check of the machines regarding whether there are supplies in shortage or to be replaced, or whether there are parts out of order.

### SUMMARY OF THE INVENTION

The present invention contrives to solve the disadvantages of the prior art.

An objective of the invention is to provide an integrated control system and method that can centrally maintain multiple automatic medicine packaging apparatuses.

Another objective of the invention is to provide an integrated control system and method that are linked to a warehouse management system for supplies for the machines that include tablets, printer ribbons and medicine envelopes, etc. and manages supplies in the warehouse.

In order to achieve the above objective, the present invention provides an integrated control system for controlling a plurality of automatic tablet packaging apparatuses. The system includes an operation unit that receives operation signal input for performing functions, a communication unit that is adapted to send/receive data to and from the automatic tablet packaging machines, and a microprocessor that stores programs that perform communication with the automatic tablet packaging apparatuses and managing tablet inventory and expendable items, controls data communication by the communication unit, and controls display of operation status of a particular automatic table packaging apparatus.

The operation unit selects one of the automatic table packaging apparatuses for which checking status is required, and receives operation signal input for checking detailed status for the appointed apparatus.

The integrated control system is adapted to communicate with a warehouse management system. The microprocessor performs managing tablets and expendable items stored in a warehouse.

The microprocessor controls operation of the automatic tablet packaging apparatus based on inventory and status data of the automatic tablet packaging apparatus and analysis of the data.

The integrated control system further includes a display control unit that controls display of inventory information and operation status for the automatic tablet packaging apparatus according to display control signal that is output from the microprocessor, and a display that is connected to the display control unit and displays the inventory and operation status for the automatic tablet packaging apparatus.

The communication unit is adapted to transmit an alert message, which is generated when abnormal operation of the automatic tablet packaging apparatus is detected, to a communication device of a user, and to transmit on site monitoring information for the automatic tablet packaging apparatus.

The integrated control system is adapted to connect with the automatic tablet packaging apparatuses with LAN, CAN, RS422 or RS485, and to connect with a communication device of a user with a wireless communication network.

The microprocessor includes a data input module that receives input of function data via the operation unit, a communication control module that is connected with the communication unit and controls data communication with the automatic tablet packaging apparatus, transmits error message and transmits on site monitoring data, a control module that controls sending/receiving of the data, tablet inventory management for the automatic tablet packaging apparatus, and storing and display of data, a packaging apparatus inventory management module that analyzes and manages tablet inventory of each automatic tablet packaging apparatus that is transmitted through the communication control module, a display control module that controls display of operation status and tablet inventory of the automatic tablet packaging apparatus according to the control of the control module, and a storage control module that controls storing data from the automatic tablet packaging apparatus or power failure check data according to the control of the control module.

The microprocessor further includes a prescription data processing module that analyzes prescription data that is transmitted through the communication control module and predicts expected tablet inventory for each kind of tablets when the packaging is done, an out-of-stock timing prediction module that analyzes inventory for each tablet that is obtained from the packaging machine inventory management module and predicts out-of-stock timing for each tablet, and an out-of-stock tablet packaging prevention module that is linked with the prescription data processing module and the packaging machine inventory management module, decides whether a tablet is out of stock, and generates control signal that forcefully terminates operation of the corresponding machine when there is a tablet that is out-of-stock.

The microprocessor further includes a printing control module that is connected to the control module and controls printing of tablet status data, a warehouse medicine inventory management module that analyzes and manages warehouse medicine inventory that is transmitted through the communication control module, and an expendable supplies management module that is connected to the control module and manages expendable supplies.

The out-of-stock timing prediction module calculates average consumption rate by dividing the weekly consumption amount of a tablet by number of days, compares the average consumption rate and current inventory of the tablet, and predicts the out-of-stock timing of the tablet.

The integrated control system further includes a power failure check unit that checks whether power for the automatic tablet packaging apparatus has failed or not, and enables error message output when a power failure occurred, and a print data processing unit that processes print data from the automatic tablet packaging apparatus, which includes hourly tablet information and inventory of tablets, and transmits the processed data to a printer.

The display is divided into a plurality of sections, each of which corresponds to each of the automatic tablet packaging apparatuses. The section displays tablet inventory and operation status for the automatic tablet packaging apparatus. The display utilizes different combinations of characters and colors to display a normal state, a check required state and an abnormal status.

The normal state is displayed in green color, and indicates there is enough tablet inventory and the operation is normal. The check-required state is displayed in yellow color and indicates that one of the tablets should be supplied or there is likelihood of malfunction, which is decided by self-diagnosis. The abnormal state is displayed in red and indicates that the apparatus is out of order or the tablet is in shortage.

The invention provides a method for integrally controlling a plurality of automatic tablet packaging apparatuses and a warehouse. The method is performed by an integrated control system that comprises an operation unit that receives operation signal input for performing functions, a communication unit that is adapted to send/receive data to and from the automatic tablet packaging apparatuses, and a microprocessor that stores programs that perform communication with the automatic tablet packaging apparatuses and a warehouse management system and managing tablet inventory and expendable items, controls data communication by the communication unit, and controls display of operation status of a particular automatic table packaging apparatus, a display control unit that controls display of inventory information and operation status for the automatic tablet packaging apparatus according to display control signal that is output from the microprocessor, and a display that is connected to the display control unit and displays the inventory and operation status for the automatic tablet packaging apparatus.

The method includes the steps of receiving input through the operation unit, communicating data via the communication unit, controlling the operation of the integrated control system, analyzing and managing of the total tablet inventory in the warehouse, analyzing and managing of tablet inventory for each of the automatic tablet packaging apparatuses, predicting tablet inventory after packaging by analyzing prescription, predicting out-of-stock timing of each tablet by analyzing tablet inventory, deciding shortage of tablet and stopping the automatic tablet packaging apparatus when there is shortage of tablet, printing of tablet inventory, managing expendable supplies by analyzing data of the warehouse management system, displaying of tablet inventory and operation status and storing data.

The data in the step of communicating data via the communication unit includes data of the automatic tablet packaging apparatus and the warehouse, alert message and on-site monitoring information. The step of controlling the operation of the integrated control system controls data communication, managing tablet inventory in the warehouse and in the automatic tablet packaging apparatuses, and storing and displaying data. The data in the step of storing data includes data from the automatic tablet packaging apparatuses and the warehouse management system and a power failure check data.

The advantages of the present invention are: (1) a single system can integrally control and manage many tablet packaging apparatuses; (2) a single operator or system manager can manage many packaging apparatuses; (3) a user can check individual apparatus' general and detail operation status with the display; (4) remote monitoring is possible via wireless communication network; (5) management of warehouse is also facilitated with information displayed on the display.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a schematic diagram for an integrated control system for automatic tablet packaging apparatuses according to the present invention;

FIG. 2 is a block diagram showing the system;

FIG. 3 is a block diagram of a microprocessor;

FIG. 4 is an illustrative view of a display; and

FIG. 5 is a flow diagram that shows a method for integrally controlling automatic tablet packaging apparatuses.

### DETAILED DESCRIPTION OF THE INVENTION

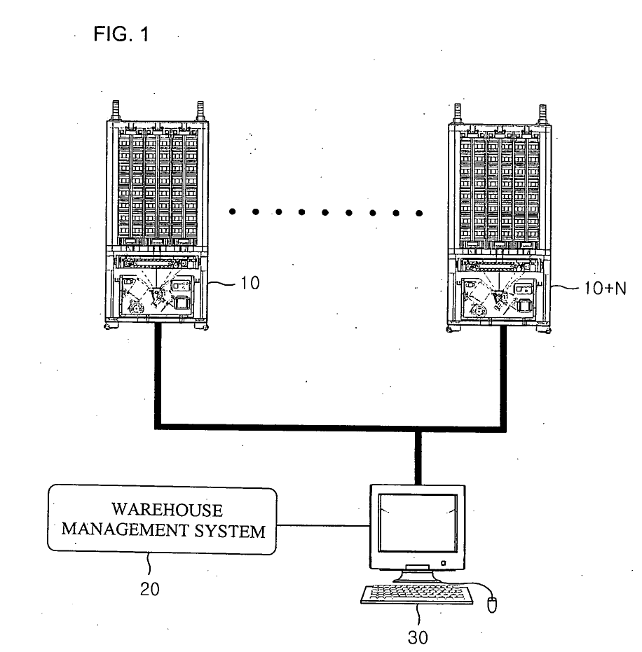

FIG. 1 shows that an integrated control system 30 is connected to a plurality of automatic tablet packaging machines 10˜10+N, and a warehouse management system 20 with LAN or CAN.

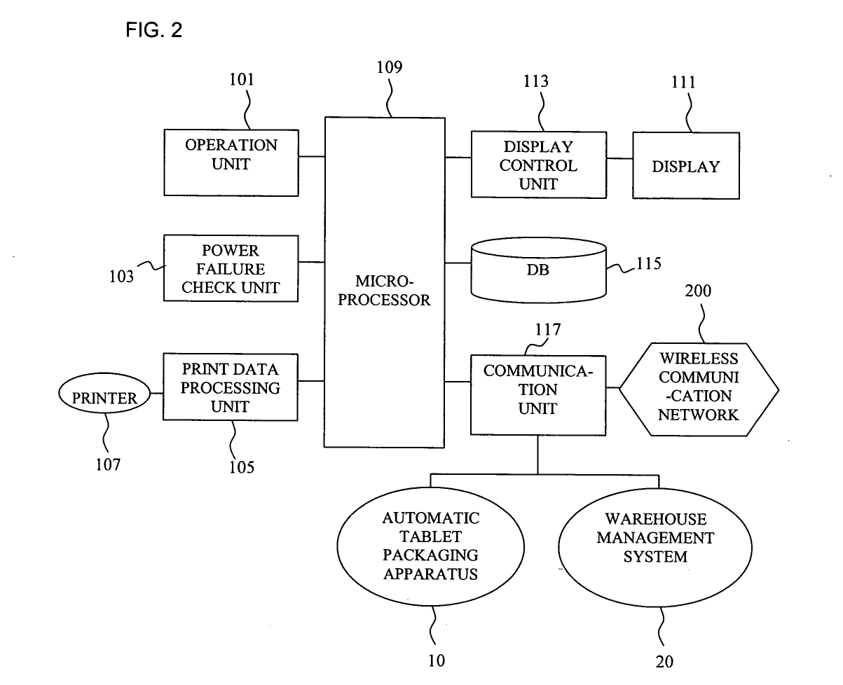

FIG. 2 shows the detail construction of the integrated control system 30. An operation unit 101 receives operation signal input for performing functions; appoints one of the automatic table packing machines for which checking status is required; and receives operation signal input for checking detailed status for the appointed machine. A power failure check unit 103 checks whether power for the machine has failed or not, and enables error message output when a power failure occurred.

A print data processing unit 105 processes print data from the automatic tablet packaging machine 10 such as hourly tablet information, and inventory of tablets, and transmits the processed data to a printer 107. A communication unit 117 sends/receives data to and from the automatic tablet packaging machines 10˜10+N, through LAN, CAN, RS422, RS485, etc. The communication unit 117 is also connected to a wireless communication network 200 and transmits error messages when the automatic tablet packaging machine 10 become out of order, and monitoring data regarding on site situation to a wireless phone of a user (prescription administrator). In addition, the communication unit 117 internally communicates with the warehouse management system 20 that manages a warehouse which stores tablets and other expendable items.

A microprocessor 109 stores programs that perform communication with the automatic tablet packaging machines, and managing tablet inventory and expendable items; controls data communication with the communication unit 117; and controls display of situation of a particular automatic table packaging machine and operation of the machine based on inventory and status data of the machines obtained through the communication unit 117 and analysis of the data. In addition, the microprocessor 109 performs the function of managing tablets and expendable items stored in the warehouse via communication with the warehouse management system 20.

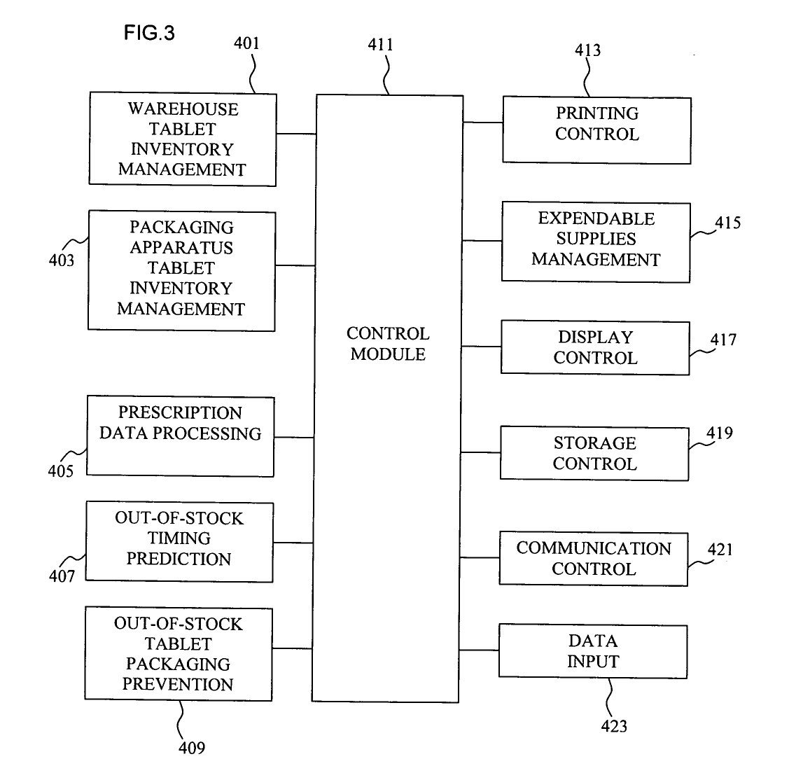

FIG. 3 shows that the microprocessor 109 includes a data input module 423 that receives input of function data, etc. via the operation unit 101; a communication control module 421 that is connected with the communication unit 117, and controls data communication with the automatic tablet packaging machine 10, transmitting error message and transmitting on site monitoring data; and a packaging machine inventory management module 403 that analyzes and manages tablet inventory of each automatic tablet packaging machine that is transmitted through the communication control module 421.

The microprocessor 109 further includes a prescription data processing module 405 that analyzes prescription data that is transmitted through the communication control module 421 and predicts expected tablet inventory for each kind of tablets when the packaging is done; an out-of-stock timing prediction module 407 that analyzes inventory for each tablet that is obtained from the packaging machine inventory management module 403 and predicts out-of-stock timing for each tablet; and an out-of-stock tablet packaging prevention module 409 that is linked with the prescription data processing module 405 and the packaging machine inventory management module 403, decides whether a tablet is out of stock, and generates control signal that forcefully terminates operation of the corresponding machine when there is a tablet that is out-of-stock.

The microprocessor 109 further includes a control module 411 that controls sending/receiving of the data, tablet inventory management for the automatic tablet packaging machine and storing and display of data; a printing control module 413 that is connected to the control module 411 and controls printing of tablet status data; a display control module 417 that controls display of the normal operation status of the automatic tablet packaging apparatus and tablet inventory etc. according to the control of the control unit 411, and a storage control module 419 that controls storage data from the automatic tablet packaging machine and power failure check data, etc. according to the control of the control module 411.

The microprocessor 109 further includes a warehouse medicine inventory management module 401 that analyzes and manages warehouse medicine inventory that is transmitted through the communication control module 421, and an expendable supplies management module 415 that is connected to the control module 411 and manages expendable supplies that includes printer ribbons, medicine envelopes, etc.

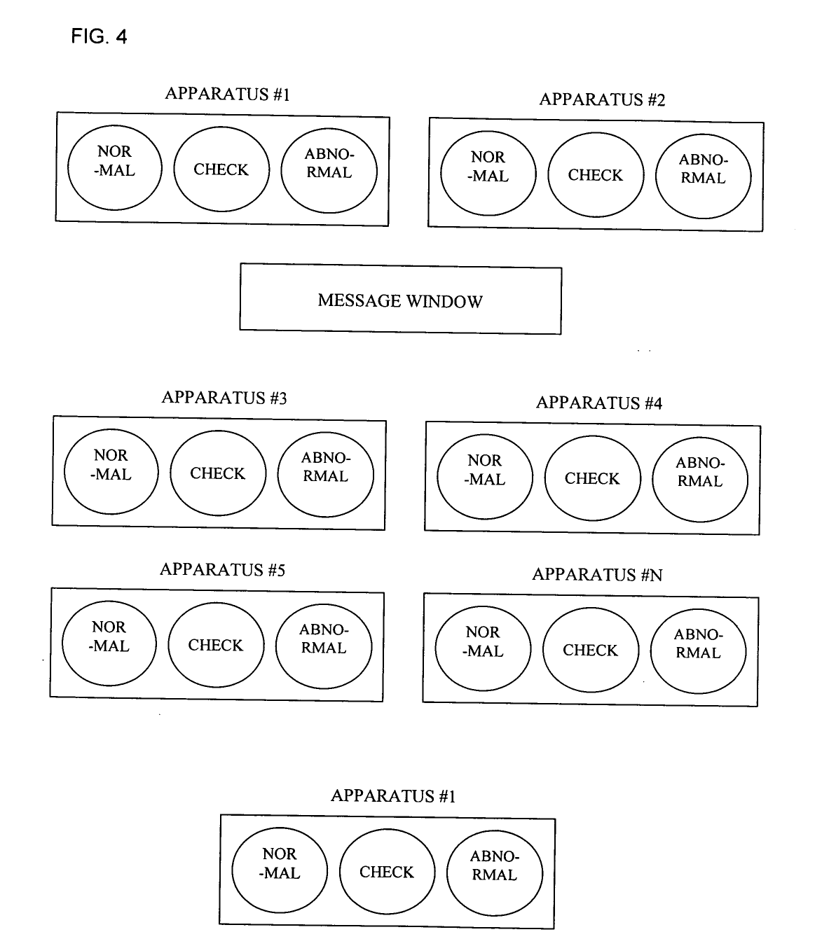

The display control unit 113 controls display of inventory for each tablet, printing ribbon, medicine envelope, and normal operation status of the packaging device and the power for the automatic medicine packaging machine according to display control signal that is output from the microprocessor 109. The display control unit 113 also is connected to the warehouse management system and controls display of the total inventory. FIG. 4 shows a display 111 that is connected to the display control unit 113 and displays the inventory and operation status for each automatic medicine packaging apparatus and the total inventory of the warehouse.

The display 111 is partitioned for each of the automatic tablet packaging machines by unique numbers. When a machine becomes out of order, the partition for the corresponding display changes color from green to orange or red depending on the severity and urgency of the situation, and displays error message. Also, the machine in out of order turns on an indicator light that is provided on top of the machine and generates alarm sound. The display 111 may selectively display inventory data for a particular machine such as tablet inventory, remaining amount of print ribbons and medicine envelopes, etc. when the machine is selected, and total tablet and expendable supplies inventory when the warehouse management system is selected. The display 111 displays error message when power failure or other abnormality for a machine occurs.

The operation of the integrated control system and method of the present invention is explained in detail below.

Each of the automatic medicine packaging apparatuses 10˜10+N periodically transmits its tablet inventory and normal operation status to the integrated control system 30.

The microprocessor 109 receives various data that is related to the operation of the system such as power failure check data through the data input module 423 and stores it in the database 115. The microprocessor 109 analyzes the data and if it decides that a power failure occurred for example, controls the display control module 417 to output an error message. The display control module 417 controls that a power failure message is displayed through the display control unit 113 and the display 111 displays power failure error message in this way.

The microprocessor 109 receives data including tablet inventory for each tablet cassette, remaining amount of a print ribbon that is sensed by a sensor installed in a print unit, remaining amount of medicine envelopes sensed by a sensor installed in a sealing unit and operation status data from the automatic medicine packaging apparatus via the communication control module 421. The packaging machine medicine inventory management module 403 analyzes the data from each apparatus in real time, recognizes the operation status including whether malfunction occurred and inventory for each medicine, and transfers the result to the control module 411. For example, the automatic tablet packaging machine number 1 contains A, B, C, D, E, F, G, H tablets. The inventory in a cassette is calculated by subtracting discharged amount from the initially filled amount. The discharged amount is obtained by counting dispensed amount according to prescriptions. Therefore, when the minimum table amount is preset and stored, the shortage of tablet (need to refill of tablet) may be easily determined.

In this way, the inventory of each cassette in each packaging apparatus is analyzed and stored in the database 115. The control module 411 can check the inventory and operation status of the automatic table packaging apparatus via linking with the packaging apparatus medicine inventory management module 403, and controls so that the status of the apparatus is displayed in real time with the display control unit 113.

Referring to FIG. 4, the display 111 is divided into a plurality of sections, each of which corresponds to each of the automatic medicine packaging apparatuses. The section displays tablet inventory and operation status for the apparatus. LED is preferably used for the display 111. The display 111 utilizes different combinations of characters and colors to display a normal state, a check required state and an abnormal status. The normal state is displayed in green color, and indicates there is enough tablet inventory and the operation is normal. The check-required state is displayed in yellow color and indicates that one of the tablets should be supplied or there is likelihood of malfunction, which may be decided by self-diagnosis. The abnormal state is displayed in red and indicates that the apparatus is out of order or the tablet is in shortage.

To display further detail, a user selects a particular automatic tablet packaging apparatus with the operation unit 101, and inputs information only for recognizing the status of the selected apparatus. The control module 411 recognizes this and retrieves information that the user wants to know via linking to the storage control module 419 and let the information to be displayed in a message window of the display 111. For example, when the user chose tablet shortage of apparatus number 1, the message window displays the name of the tablet that is in shortage and current inventory, etc. LCD or any other display device that can show data is used for the message window.

The status of the apparatus must be updated in real time whenever a prescription is generated. When a prescription is transmitted, the prescription data processing module 405 processes the prescription data, and the tablet inventory after completion of packaging is predicted by comparing the current inventory and the tablet information of the prescription data. The data analyzed and predicted in this way is stored in the database 115.

The integrated control system 30 can predict the timing of the tablet shortage. The out-of-stock timing prediction module 407 calculates average consumption rate by dividing the weekly consumption amount of a table by number of days, compares the average consumption rate and current inventory of the tablet, and predicts the out-of-stock timing of the tablet. The timing is shown with the display 111 and the user may know the tablet which is predicted to be out of stock soon and purchases the tablet. In this way, failure of fulfilling prescription order is prevented, and systematic purchase of tablets is possible.

The system prevents packaging without medicine that is out of stock. Manual intervention was required in the past. When a tablet shortage occurs in a particular apparatus, the out-of-stock tablet packaging prevention module 409 links with the control module 411 and the prescription data processing module 405 and sends command to the apparatus via the communication control module 421 so that the apparatus forcefully stops packaging operation in response to the command. The display 111 displays this situation.

The microprocessor 109 can print data that the user requests via the printing control module 413. The user can check status or history information etc. with printouts provided by the printer 107.

When the user is away from the integrated control system 30, she cannot check the display 111 with her eye.

When an action is not taken within a predetermined time (for example, 10 minutes) after the microprocessor 109 made the display 111 show abnormal state, table shortage, etc., the microprocessor 109 sends alert message to an wireless communication device that the user carries, for example, a cell phone via the communication control module 421 and the communication unit 117 transmits the alert message via the wireless communication network 200.

In this way, even when the user is away from the integrated control system 30, the user is kept informed of any abnormal status.

The integrated control system enables remote system monitoring. When a command is input to the operation unit 101, the microprocessor 109 stores it in the database 115 and changes the mode of the system. In the remote system monitoring mode, the on site situation of each automatic tablet packaging apparatus is photographed with a camera (not shown), and the photograph is transmitted periodically or nonperiodically to the cell phone of the user via the wireless communication network 200. In this way, the user can monitor the on site situation with her cell phone.

The integrated control system 30 manages the warehouse by linking with the warehouse management system 20.

The warehouse management system 20 transmits the total tablet inventory and expendable supplies inventory to the integrated control system 30 periodically or nonperiodically.

The warehouse medicine inventory management module 401 analyzes the received information to figure out inventory status. The analyzed warehouse information is stored in the database 115 via the storage control module 419.

When a shortage or shortage prediction is decided upon analysis of the warehouse inventory data, the control module 411 is informed of the situation and the control module 411 controls the storage control module 419 to store the information, and controls the display control module 419 to display the situation or timing on the display 111.

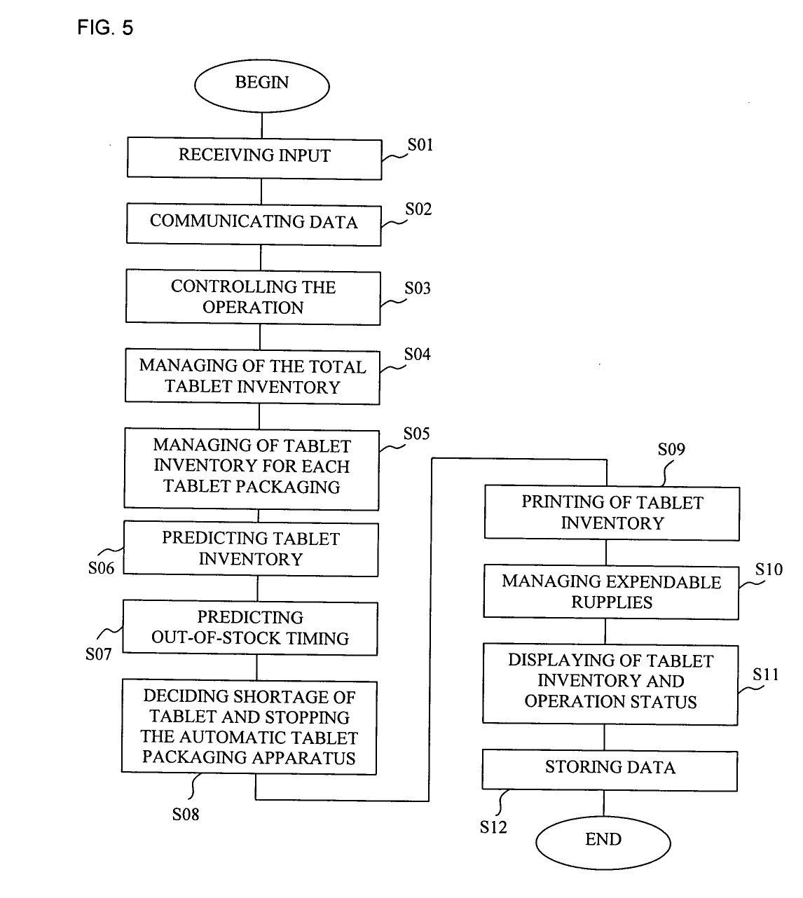

FIG. 5 shows a method for integrally controlling a plurality of automatic tablet packaging apparatuses and a warehouse. The method is performed by the integrated control system 30.

The method includes step S01 of receiving input through the operation unit, step S02 of communicating data via the communication unit, step S03 of controlling the operation of the integrated control system, step S04 of analyzing and managing of the total tablet inventory in the warehouse, step S05 of analyzing and managing of tablet inventory for each of the automatic tablet packaging apparatuses, step S06 of predicting tablet inventory after packaging by analyzing prescription, step S07 of predicting out-of-stock timing of each tablet by analyzing tablet inventory, step S08 of deciding shortage of tablet and stopping the automatic tablet packaging apparatus when there is shortage of tablet, step S09 of printing of tablet inventory, step S10 of managing expendable supplies by analyzing data of the warehouse management system, step S11 of displaying of tablet inventory and operation status and step S12 of storing data.

The data in step S02 includes data of the automatic tablet packaging apparatus and the warehouse, alert message and on-site monitoring information. Step S03 controls data communication, managing tablet inventory in the warehouse and in the automatic tablet packaging apparatuses, and storing and displaying data. The data in Step S12 includes data from the automatic tablet packaging apparatuses and the warehouse management system and a power failure check data.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. An integrated control system for controlling a plurality of automatic tablet packaging apparatuses, comprising:
a) an operation unit that receives operation signal input for performing functions;
b) a communication unit that is adapted to send/receive data to and from the automatic tablet packaging machines; and
c) a microprocessor that stores programs that perform communication with the automatic tablet packaging apparatuses and managing tablet inventory and expendable items, controls data communication by the communication unit, and controls display of operation status of a particular automatic table packaging apparatus.

2. The integrated control system of claim 1, wherein the operation unit appoints one of the automatic table packaging apparatuses for which checking status is required, and receives operation signal input for checking detailed status for the appointed apparatus.

3. The integrated control system of claim 1, wherein the integrated control system is adapted to communicate with a warehouse management system, wherein the microprocessor performs managing tablets and expendable items stored in a warehouse.

4. The integrated control system of claim 1, wherein the microprocessor controls operation of the automatic tablet packaging apparatus based on inventory and status data of the automatic tablet packaging apparatus and analysis of the data.

5. The integrated control system of claim 1, further comprising a display control unit that controls display of inventory information and operation status for the automatic tablet packaging apparatus according to display control signal that is output from the microprocessor, and a display that is connected to the display control unit and displays the inventory and operation status for the automatic tablet packaging apparatus.

6. The integrated control system of claim 1, wherein the communication unit is adapted to transmit an alert message, which is generated when abnormal operation of the automatic tablet packaging apparatus is detected, to a communication device of a user.

7. The integrated control system of claim 1, wherein the communication unit is adapted to transmit on site monitoring information for the automatic tablet packaging apparatus.

8. The integrated control system of claim 1, wherein the integrated control system is adapted to connect with the automatic tablet packaging apparatuses with LAN, CAN, RS422 or RS485.

9. The integrated control system of claim 1, wherein the integrated control system is adapted to connect with a communication device of a user with a wireless communication network.

10. The integrated control system of claim 1, wherein the microprocessor comprises:
a) a data input module that receives input of function data via the operation unit;
b) a communication control module that is connected with the communication unit and controls data communication with the automatic tablet packaging apparatus, transmits error message and transmits on site monitoring data;
c) a control module that controls sending/receiving of the data, tablet inventory management for the automatic tablet packaging apparatus, and storing and display of data;
d) a packaging apparatus inventory management module that analyzes and manages tablet inventory of each automatic tablet packaging apparatus that is transmitted through the communication control module;
e) a display control module that controls display of operation status and tablet inventory of the automatic tablet packaging apparatus according to the control of the control module; and
f) a storage control module that controls storing data from the automatic tablet packaging apparatus or power failure check data according to the control of the control module.

11. The integrated control system of claim 10, wherein the microprocessor further comprises:
a) a prescription data processing module that analyzes prescription data that is transmitted through the communication control module and predicts expected tablet inventory for each kind of tablets when the packaging is done;
b) an out-of-stock timing prediction module that analyzes inventory for each tablet that is obtained from the packaging machine inventory management module and predicts out-of-stock timing for each tablet; and
c) an out-of-stock tablet packaging prevention module that is linked with the prescription data processing module and the packaging machine inventory management module , decides whether a tablet is out of stock, and generates control signal that forcefully terminates operation of the corresponding machine when there is a tablet that is out-of-stock.

12. The integrated control system of claim 10, wherein the microprocessor further comprises:
a) a printing control module that is connected to the control module and controls printing of tablet status data;
b) a warehouse medicine inventory management module that analyzes and manages warehouse medicine inventory that is transmitted through the communication control module; and
c) an expendable supplies management module that is connected to the control module and manages expendable supplies.

13. The integrated control system of claim 1, wherein the microprocessor comprises:
a) a prescription data processing module that analyzes prescription data that is transmitted through the communication control module and predicts expected tablet inventory for each kind of tablets when the packaging is done;
b) an out-of-stock timing prediction module that analyzes inventory for each tablet that is obtained from the packaging machine inventory management module and predicts out-of-stock timing for each tablet; and
c) an out-of-stock tablet packaging prevention module that is linked with the prescription data processing module and the packaging machine inventory management module, decides whether a tablet is out of stock, and generates control signal that forcefully terminates operation of the corresponding machine when there is a tablet that is out-of-stock.

14. The integrated control system of claim 13, wherein the out-of-stock timing prediction module calculates average consumption rate by dividing the weekly consumption amount of a tablet by number of days, compares the average consumption rate and current inventory of the tablet, and predicts the out-of-stock timing of the tablet.

15. The integrated control system of claim 1, wherein the microprocessor comprises:
a) a printing control module that is connected to the control module and controls printing of tablet status data;
b) a warehouse medicine inventory management module that analyzes and manages warehouse medicine inventory that is transmitted through the communication control module; and
c) an expendable supplies management module that is connected to the control module and manages expendable supplies.

16. The integrated control system of claim 1, further comprising:
a) a power failure check unit that checks whether power for the automatic tablet packaging apparatus has failed or not, and enables error message output when a power failure occurred; and
b) a print data processing unit that processes print data from the automatic tablet packaging apparatus, which includes hourly tablet information and inventory of tablets, and transmits the processed data to a printer.

17. The integrated control system of claim 1, wherein the display is divided into a plurality of sections, each of which corresponds to each of the automatic tablet packaging apparatuses, wherein the section displays tablet inventory and operation status for the automatic tablet packaging apparatus, wherein the display utilizes different combinations of characters and colors to display a normal state, a check required state and an abnormal status.

18. The integrated control system of claim 17, wherein the normal state is displayed in green color, and indicates there is enough tablet inventory and the operation is normal, wherein the check-required state is displayed in yellow color and indicates that one of the tablets should be supplied or there is likelihood of malfunction, which is decided by self-diagnosis, wherein the abnormal state is displayed in red and indicates that the apparatus is out of order or the tablet is in shortage.

19. A method for integrally controlling a plurality of automatic tablet packaging apparatuses and a warehouse, wherein the method is performed by an integrated control system that comprises an operation unit that receives operation signal input for performing functions, a communication unit that is adapted to send/receive data to and from the automatic tablet packaging apparatuses; and a microprocessor that stores programs that perform communication with the automatic tablet packaging apparatuses and a warehouse management system and managing tablet inventory and expendable items, controls data communication by the communication unit, and controls display of operation status of a particular automatic table packaging apparatus, a display control unit that controls display of inventory information and operation status for the automatic tablet packaging apparatus according to display control signal that is output from the microprocessor, and a display that is connected to the display control unit and displays the inventory and operation status for the automatic tablet packaging apparatus, the method comprising the steps of:
a) receiving input through the operation unit;
b) communicating data via the communication unit;
c) controlling the operation of the integrated control system;
d) analyzing and managing of the total tablet inventory in the warehouse;
e) analyzing and managing of tablet inventory for each of the automatic tablet packaging apparatuses;
f) predicting tablet inventory after packaging by analyzing prescription;
g) predicting out-of-stock timing of each tablet by analyzing tablet inventory;
h) deciding shortage of tablet and stopping the automatic tablet packaging apparatus when there is shortage of tablet;
i) printing of tablet inventory;
j) managing expendable supplies by analyzing data of the warehouse management system;
k) displaying of tablet inventory and operation status; and
l) storing data.

20. The method of claim 19, wherein the data in the step of communicating data via the communication unit includes data of the automatic tablet packaging apparatus and the warehouse, alert message and on-site monitoring information, wherein the step of controlling the operation of the integrated control system controls data communication, managing tablet inventory in the warehouse and in the automatic tablet packaging apparatuses, and storing and displaying data, wherein the data in the step of storing data includes data from the automatic tablet packaging apparatuses and the warehouse management system and a power failure check data.
